(19) United States
(12) Patent Application Publication
(10) Pub. No.: US 2015/0208760 A1
(43) Pub. Date: Jul. 30, 2015

(54) SOLE FOR REHABILITATION FOOTWEAR

(71) Applicant: Tung-Cheng CHEN, Taichung City (TW)

(72) Inventor: Tung-Cheng CHEN, Taichung City (TW)

(21) Appl. No.: 14/162,960

(22) Filed: Jan. 24, 2014

(51) Int. Cl. A43B 13/20; A61F 5/01

(52) U.S. Cl. CPC A43B 13/20; A61F 5/0195

(57) ABSTRACT

A sole for rehabilitation footwear has a body and a shock-absorbing layer. The body has a connecting frame and a holding groove. The holding groove is formed in a bottom of the body along a periphery of the body between the connecting frame and a bottom surface of the bottom of the body. The shock-absorbing layer is connected to the body and has a mounting flange. The mounting flange is annularly formed on and protrudes from a top surface of the shock-absorbing layer along a periphery of the shock-absorbing layer and is mounted in the holding groove of the body.

### BACKGROUND OF THE INVENTION

1. Field of the Invention

The present invention relates to a sole, and more particularly to a sole for rehabilitation footwear that can provide a preferred rehabilitation effect to a patient.

2. Description of Related Art

Conventional rehabilitation footwear has a sole, a supporting frame and multiple fastening belts. The sole has a body and a shock-absorbing layer. The body has a flat bottom. The shock-absorbing layer is securely mounted on the flat bottom of the body to fully abut the ground. The supporting frame is connected to the body of the sole to support the leg of a patient. The fastening belts are connected to the body of the sole and the supporting frame to hold the patient's leg and foot with the conventional rehabilitation footwear.

However, the conventional rehabilitation footwear can provide a protecting and rehabilitating effect to the patient's injured leg and foot. When the patent uses the conventional rehabilitation footwear and walks, a front end of the shock-absorbing layer may be lifted off by the friction with the ground due to the shock-absorbing layer is mounted on the flat bottom of the body of the sole. When the shock-absorbing layer is lifted, the patient may be got a secondary hurt by the raising shock-absorbing layer and this is unsafe in use.

The present invention provides a sole for rehabilitation footwear to mitigate or obviate the aforementioned problems.

### SUMMARY OF THE INVENTION

The main objective of the present invention is to provide a sole for rehabilitation footwear that can provide a preferred rehabilitation effect to a patient.

The sole for rehabilitation footwear in accordance with the present invention has a body and a shock-absorbing layer. The body has a connecting frame and a holding groove. The holding groove is formed in a bottom of the body along a periphery of the body between the connecting frame and a bottom surface of the bottom of the body. The shock-absorbing layer is connected to the body and has a mounting flange. The mounting flange is annularly formed on and protrudes from a top surface of the shock-absorbing layer along a periphery of the shock-absorbing layer and is mounted in the holding groove of the body.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a sole for rehabilitation footwear in accordance with the present invention, is connected to a supporting frame and multiple fastening belts of rehabilitation footwear;

FIG. 2 is an enlarged exploded perspective top view of the sole in FIG. 1;

FIG. 3 is an enlarged exploded perspective bottom view of the sole in FIG. 1;

FIG. 4 is an enlarged side view of the sole in FIG. 1; and

FIG. 5 is an enlarged and cross sectional side view of the sole in FIG. 4.

### DETAILED DESCRIPTION OF THE PREFERRED EMBODIMENT

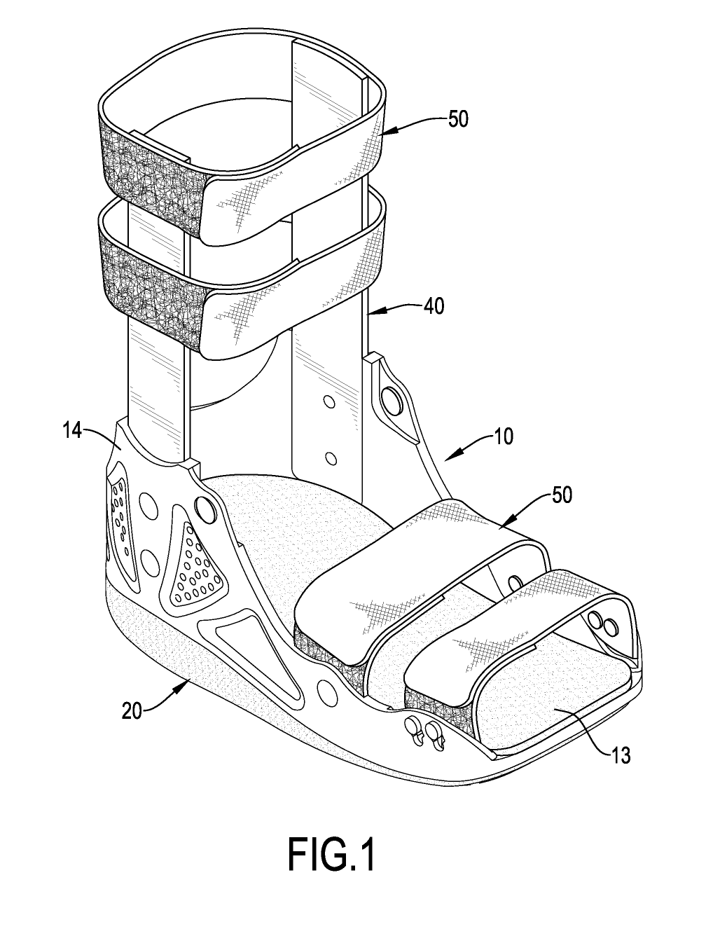

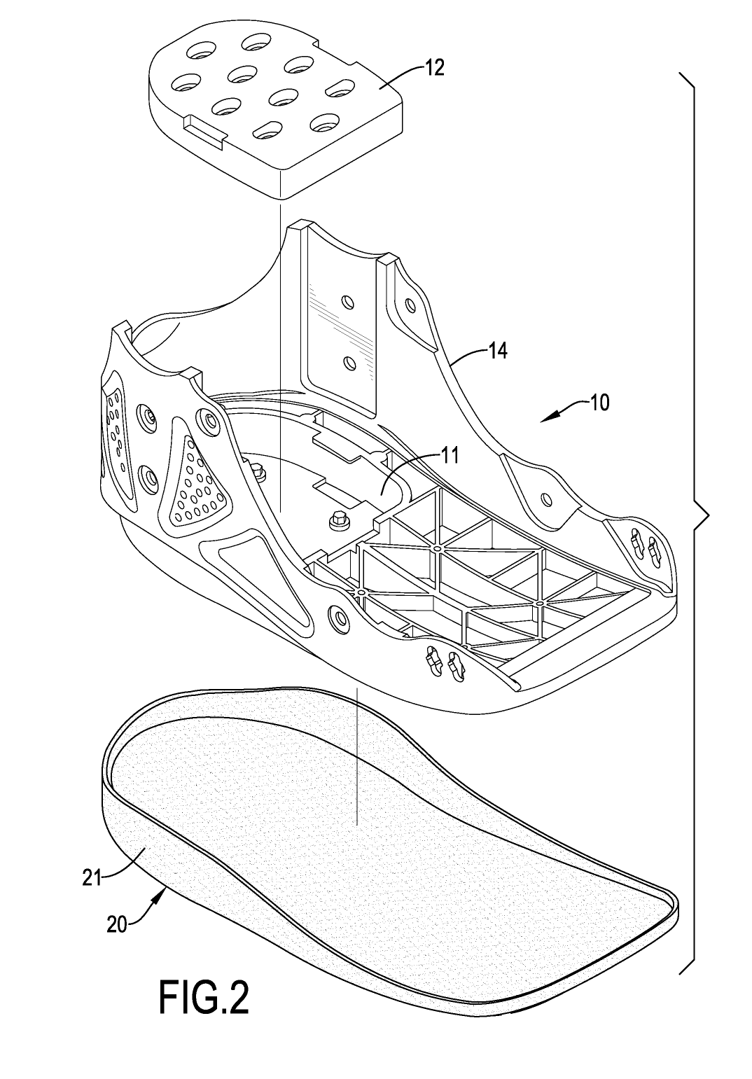

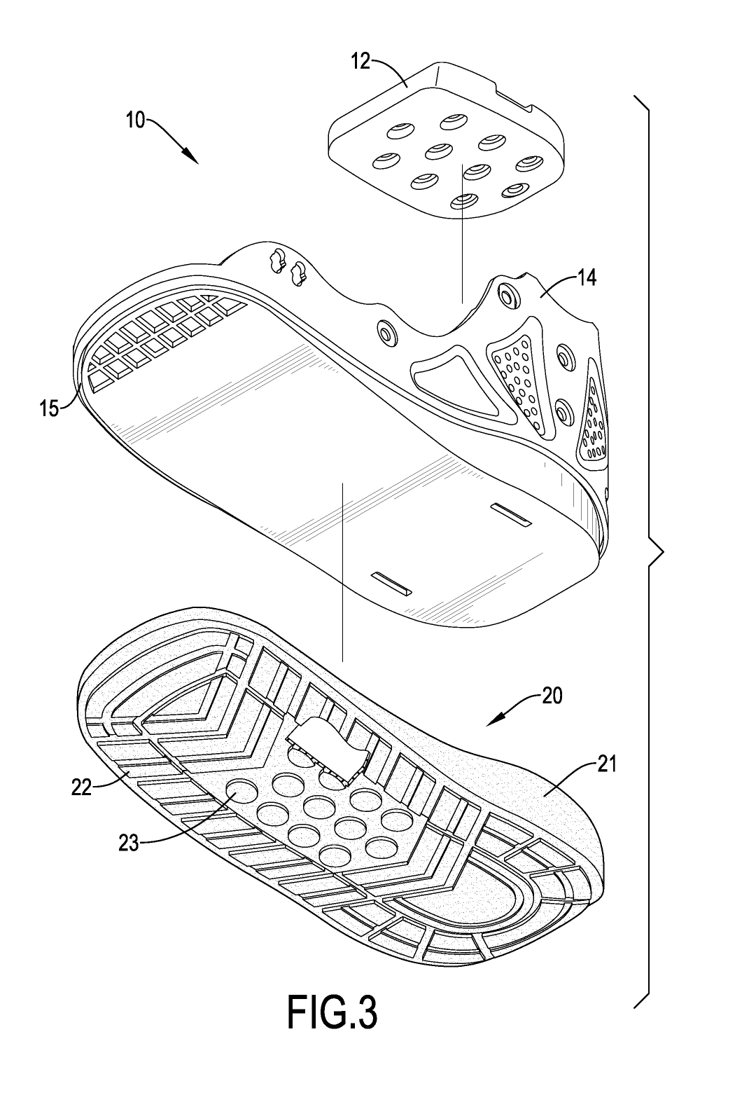

With reference to FIGS. 1 to 3, a sole for rehabilitation footwear in accordance with the present invention has a body 10 and a shock-absorbing layer 20.

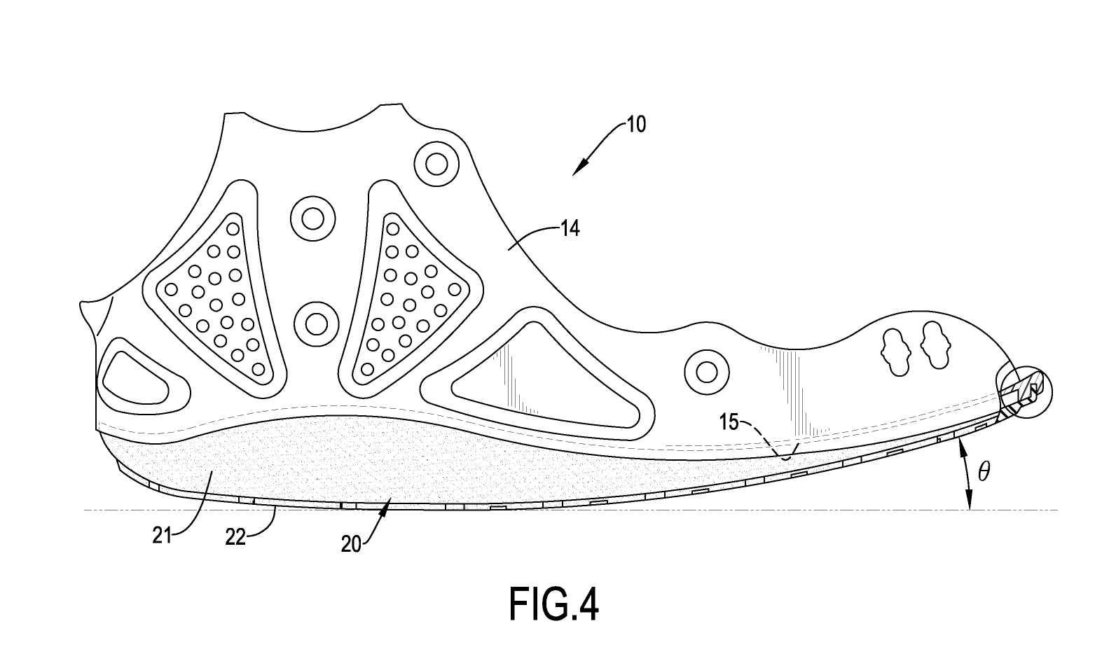

The body 10 has a rear end, a front end, a top surface, a periphery, a bottom, a mounting recess 11, an air cushion 12, a pad 13, a connecting frame 14 and a holding groove 15. With reference to FIG. 4, the front end of the body 10 is formed with the rear end of the body 10 and is curved upwardly to have an angle (θ) relative to the ground. That is, the bottom surface of the body 10 at the front end of the body 10 does not abut the ground. The mounting recess 11 is formed in the top surface of the body 10 at the rear end of the body 10. The air cushion 12 is mounted in the mounting recess 11 of the body 10.

The pad 13 is mounted on the top surface of the body 10 to cover the air cushion 12. The connecting frame 14 is formed on and protrudes upwardly from the top surface of the body along the periphery of the body 10. The holding groove 15 is formed in the bottom of the body 10 along the periphery of the body 10 between the connecting frame 14 and a bottom surface of the bottom of the body 10.

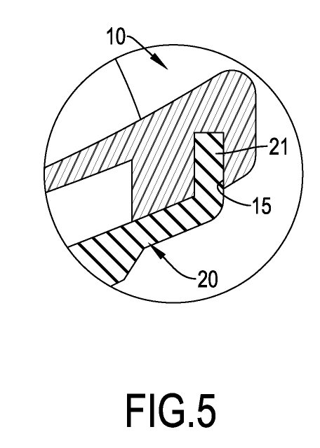

The shock-absorbing layer 20 is connected to the body 10 and has a top surface, a bottom surface, a periphery, a mounting flange 21, multiple skidproof protrusions 22 and multiple sucking protrusions 23. The top surface of the shock-absorbing layer 20 abuts the bottom face of the bottom of the body 10. With reference to FIGS. 4 and 5, the mounting flange 21 is annularly formed on and protrudes from the top surface of the shock-absorbing layer 20 along the periphery of the shock-absorbing layer 20 and is mounted in the holding groove 15 of the body 10. Additionally, an adhesive face is defined between the top surface of the shock-absorbing layer 20 and an inner surface of the mounting flange 21.

The skidproof protrusions 22 are formed on and protrude from the bottom surface of the shock-absorbing layer 20 at intervals. Preferably, the intervals between the skidproof protrusions 22 are the same. The sucking protrusions 23 are formed on and protrude from the bottom surface of the shock-absorbing layer 20 between the skidproof protrusions 22. In addition, each one of the sucking protrusions 23 has a curved bottom surface to enable the sucking protrusion 23 to have a sucking effect to the shock-absorbing layer 20 when the sucking protrusion 23 abuts the ground.

In use, with reference to FIG. 1, the sole is connected to a supporting frame 40 and multiple fastening belts 50 to form rehabilitation footwear, the supporting frame 40 is connected to the connecting frame 14 and the fastening belts 50 are connected to the supporting frame 40 and the connecting frame 14. When a patient uses the rehabilitation footwear in accordance with the present invention, the patient's foot is mounted on the pad 13 and the patient's leg is held with the supporting frame 40 by the fastening belts 50.

When the patient walks on the ground with the rehabilitation footwear, the front end of the body 10 is curved upwardly with an angle θ relative to the ground and this can prevent the shock-absorbing layer 20 from abutting against the ground at the front end of the body 10. Then, the shock-absorbing layer 20 can be securely mounted on the body 10 without raising by the ground and this can avoid the patient getting a secondary hurt.

Furthermore, the mounting flange 21 is inserted into and is mounted in the holding groove 15 of the body 10 to enable the shock-absorbing layer 20 to mount securely on the body 10, and this can prevent the shock-absorbing layer 20 from raising when abuts against the ground at the front end of the body 10. Additionally, in assembly, the inserting type of the mounting flange 21 can enable the shock-absorbing layer 20 to align with the body 10 easily.

In addition, the adhesive face that is defined between the top surface of the shock-absorbing layer 20 and an inner surface of the mounting flange 21 can be used to increase the connecting area between the body 10 and the shock-absorbing layer 20, and this can increase the connecting strength between the body 10 and the shock-absorbing layer 20.

Additionally, the skidproof protrusions 22 of the shock-absorbing layer 20 can provide a skidproof effect to the rehabilitation footwear and this can enable the patient to walk safely. Moreover, the sucking protrusions 23 of the shock-absorbing layer 20 also can a skidproof effect to the rehabilitation footwear and this can enable the patient to walk safely.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A sole for rehabilitation footwear comprising:
a body having
a rear end;
a front end;
a top surface;
a periphery;
a bottom having a bottom surface;
a connecting frame formed on and protruding upwardly from the top surface of the body along the periphery of the body; and
a holding groove formed in the bottom of the body along the periphery of the body between the connecting frame and the bottom surface of the bottom of the body; and
a shock-absorbing layer connected to the body and having
a top surface abutting the bottom face of the bottom of the body;
a bottom surface;
a periphery; and
a mounting flange annularly formed on and protruding from the top surface of the shock-absorbing layer along the periphery of the shock-absorbing layer and mounted in the holding groove of the body.

2. The sole for rehabilitation footwear as claimed in claim 1, wherein the front end of the body is formed with the rear end of the body and is curved upwardly to have an angle relative to the ground.

3. The sole for rehabilitation footwear as claimed in claim 2, wherein the shock-absorbing layer has an adhesive face defined between the top surface of the shock-absorbing layer and an inner surface of the mounting flange.

4. The sole for rehabilitation footwear as claimed in claim 3, wherein the shock-absorbing layer has multiple skidproof protrusions formed on and protruding from the bottom surface of the shock-absorbing layer at intervals.

5. The sole for rehabilitation footwear as claimed in claim 4, wherein the shock-absorbing layer has multiple sucking protrusions formed on and protruding from the bottom surface of the shock-absorbing layer between the skidproof protrusions.

6. The sole for rehabilitation footwear as claimed in claim 5, wherein the body has
a mounting recess formed in the top surface of the body at the rear end of the body; and
an air cushion mounted in the mounting recess of the body.

7. The sole for rehabilitation footwear as claimed in claim 6, wherein the body has a pad is mounted on the top surface of the body to cover the air cushion.

8. The sole for rehabilitation footwear as claimed in claim 1, wherein the shock-absorbing layer has an adhesive face defined between the top surface of the shock-absorbing layer and an inner surface of the mounting flange.

9. The sole for rehabilitation footwear as claimed in claim 1, wherein the shock-absorbing layer has multiple skidproof protrusions formed on and protruding from the bottom surface of the shock-absorbing layer at intervals.

10. The sole for rehabilitation footwear as claimed in claim 1, wherein the shock-absorbing layer has multiple sucking protrusions formed on and protruding from the bottom surface of the shock-absorbing layer between the skidproof protrusions.
